(19) United States
(12) Patent Application Publication
(10) Pub. No.: US 2024/0362492 A1
(43) Pub. Date: Oct. 31, 2024

(54) SYSTEMS, METHODS, AND COMPUTER-ACCESSIBLE MEDIUM FOR PROVIDING HUMAN-MODEL ALIGNMENT USING METADATA AND ARTIFACTS, PATIENT INFORMATION, OR SYNTHETIC DATA

(71) Applicant: NEW YORK UNIVERSITY, NEW YORK, NY (US)

(72) Inventor: ERIC KARL OERMANN, Chapel Hill, NC (US)

(21) Appl. No.: 18/646,234

(22) Filed: Apr. 25, 2024

Related U.S. Application Data
(60) Provisional application No. 63/461,663, filed on Apr. 25, 2023.

(51) Int. Cl. G06N 3/092; G16H 10/60

(52) U.S. Cl. CPC G06N 3/092; G16H 10/60

(57) ABSTRACT

Exemplary systems, methods, and computer-accessible medium are provided that can train a language model for a medical use or performing a medically-related procedure. Thus, exemplary systems, methods, and computer-accessible medium can be provided that can model a reward neural network on one or more physician preferences and train the language model by applying the reward neural network modeled on the physician preference(s) as feedback to guide the language model to learn the physician preference(s). The reward neural network can rely on an artificial intelligent (AI) model as a surrogate reward function for a physician feedback, and can obtain the physician preference(s) implicitly from electronic health records and/or other sources of medical data.

### CROSS REFERENCE TO RELATED APPLICATION(S)

This application relates to and claims the benefit of priority from U.S. Provisional Patent Application No. 63/461,663, filed on Apr. 25, 2023, the entire disclosure of which is incorporated herein by reference

### FIELD OF THE DISCLOSURE

The present disclosure relates generally to the methods, systems, and computer-accessible medium for providing human-model alignment using metadata and artifacts, patient information, or synthetic data.

### BACKGROUND INFORMATION

Language models have impressive capabilities, and generative language models can create compelling and creative text from human prompts. However, what makes a “good” generated text is inherently hard to define as it is subjective and context dependent. There are many applications such as writing stories where you want creativity, pieces of informative text which should be truthful, or code snippets that we want to be executable. Writing a loss function to capture these attributes can be intractable at worst, and highly inefficient at best, and most language models are still trained with a simple next token prediction loss (e.g. cross entropy). T

[symbol]

Thus, it may be beneficial to provide exemplary systems, methods and computer-accessible medium that facilitate RLAIF whereby the AI feedback is a heuristic, a weak model, constructed from cheaply available metadata and textual artifacts, that can overcome at least some of the deficiencies described herein above.

### SUMMARY OF EXEMPLARY EMBODIMENTS

To that end, it is possible to provide exemplary systems, methods and computer-accessible medium according to exemplary embodiments of the present disclosure, which can facilitate human-model alignment using metadata and artifacts, patient information, or synthetic data.

For example, to compensate for the prior shortcomings, according to an exemplary embodiment of the present disclosure, it is possible to utilize certain exemplary metrics that model human can preference, such as, e.g., BLEU or ROUGE benchmarked against reference text. On a more advanced level, e.g., it is possible to directly model human preferences using a separate reward neural network, and subsequently use the reward network to guide the generative network in optimizing for human preferences. Using the exemplary embodiments of the present disclosure, it is possible to utilize such exemplary procedure, known as Reinforcement Learning from Human Feedback (RLHF), combined with exemplary methods from a reinforcement learning to directly optimize a language model with human feedback. RLHF has facilitated language models to begin to align a model trained on a general corpus of text data to that of complex human values.

RLHF's recent success was its use in ChatGPT. However, in the medical domain, RLHF may have a shortcoming of requiring physician feedback, which can be difficult or costly to obtain in many situations. This can similarly be performed with the an artificial intelligence (AI) model providing the feedback, so called Reinforcement Learning from AI Feedback (RLAIF) if there exists an AI model configured to act as a surrogate reward function. According to various exemplary embodiments of the present disclosure, alternative procedures and/or models to RLHF can be provided and utilized which are different from costly physician preferences. For example, instead of obtaining explicit physician preferences for training a reward model, the systems, methods and computer-accessible medium according to the exemplary embodiments of the present disclosure can obtain implicit (e.g., weak) physician preferences derived from an Electronic Health Record (EHR), including, e.g., clinical data, metadata, or artifacts. Physician-EHR interactions can be important for medical care, and EHR logging of physician interactions can provide a way to develop a weak model of physician preference and, subsequently, bootstrapping a reward model for RLHF or even direct supervised training.

RLHF is an important part of of InstructGPT and ChatGPT. Existing applications rely on human feedback, obtained by large numbers of volunteers. However, it is believed that there is nothing in the prior art describing the use of EHR data broadly, and certainly more narrowly, EHR artifacts and metadata to obtain a weak, surrogate signal of physician preference, which can be performed using the systems, methods and computer-accessible medium according to the exemplary embodiments of the present disclosure.

To that end, exemplary systems, methods, and computer accessible medium according to certain exemplary embodiments of the present disclosure can be provided which can can facilitate and/or perform the training a language model for medical use by modeling a reward neural network on physician preferences and training the language model by applying the reward neural network modeled on physician preferences as feedback to guide the language model to learn physician preferences.

Furthermore, an exemplary reward neural network can rely on an AI model as a surrogate reward function for physician feedback, and the physician preferences can be obtained from the AI model providing physician preference feedback. Modeling the reward neural network can also include obtaining implicit physician preferences. These implicit physician preferences can be derived from one or more of a group comprising: electronic health records, metadata, and artifacts. The implicit physician preferences can be inferred from physician written text in the electronic health records, metadata, and artifacts.

In some exemplary embodiments of the present disclosure, the reward neural network can be finetuned on one or more ground truth notes in electronic health records to approximate the language and thinking of physicians.

These and other objects, features and advantages of the exemplary embodiments of the present disclosure will become apparent upon reading the following detailed description of the exemplary embodiments of the present disclosure, when taken in conjunction with the accompanying claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 shows a block diagram of an exemplary embodiment of a system according to the present disclosure; and

FIG. 2 shows a flowchart for an exemplary embodiment according to the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXAMPLE EMBODIMENTS

The following description of exemplary embodiments provides non-limiting representative examples referencing numerals to particularly describe features and teachings of different aspects of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

The exemplary systems, methods and computer-accessible medium according to exemplary embodiments of the present disclosure can overcome the deficiency of the prior systems and methods where discriminative and generative language models output results that are not consistent with physician preference or practice. Human-Artificial Intelligence (AI) alignment is a major challenge, particularly with language models, and existing approaches have notable drawbacks, the largest of which is the cost associated with obtaining physician preferences and the technical intractability of supervised pre-training. The exemplary systems, methods and computer-accessible medium according to exemplary embodiments of the present disclosure can eliminate the need for substantial amounts of physician data, and facilitate optimizing and aligning these models purely by observing physician interactions with the EHR. The use of patient information, in the EHR or even outside of it (conversations), can be used to align the large language model (LLM) with patients. Such patient-aligned LLMs subsequently may be a critical component of a novel type of patient simulator based on large, generative language models. This LLM can be pre-conditioned using prompt engineering, fine tuning, or reinforcement learning from human feedback (RLHF) in order to calibrate it to a specific scenario and a specific patient encounter. Such a simulator may be used in medical education, licensing, accreditation, or any other situation where simulated patients or testing is involved medicine.

An exemplary application of the exemplary systems, methods and computer-accessible medium according to exemplary embodiments of the present disclosure can be used to encourage and/or facilitate LLMs to perform the intended actions by physicians (physician-AI alignment). The exemplary systems, methods and computer-accessible medium according to exemplary embodiments of the present disclosure can utilize metadata and/or synthetic data as a way to bootstrap RLHF or direct supervised fine-tuning of LLMs to bring them to better human-LLM alignment (e.g., to align them with the physicians).

The exemplary systems, methods and computer-accessible medium according to the exemplary embodiments of the present disclosure can also utilize a number of (e.g., 3) major sources of EHR data for this purpose: clinical data, metadata, and artifacts. The exemplary clinical data can include orders placed or cancelled, notes entered, etc. Metadata can include auxiliary data to the clinical record. For example a physician addending a note may indicate that the original note was incomplete in some way (and therefore should be assigned less reward by the reward model). Artifactual data can include data logged as part of the EHR interaction process such as physician clicks, view times, and other data points captured by certain EHRs.

The exemplary systems, methods and computer-accessible medium according to exemplary embodiments of the present disclosure can further utilize such exemplary EHR data sources for an exemplary alignment either directly for fitting a reward model and/or for training a model (e.g., a weak model) of physician preferences, and then fitting a reward model to the weak preference model. It is also possible to directly perform supervised fine tuning using the metadata/artifacts as an additional label.

Exemplary aligned models according to various exemplary embodiments of the present disclosure can be used as patient simulators for education, testing, or licensing with the exemplary systems, methods and computer-accessible medium according to exemplary embodiments of the present disclosure.

Exemplary clinician aligned models according to various exemplary embodiments of the present disclosure can be used to interact with patients, automate basic medical tasks, or perform other non-critical (or maybe even critical) roles that physicians typically perform. It is further possible, according to additional exemplary embodiments of the present disclosure exemplary synthetic data to generate responses to a prompt that are labelled as e.g., not preferred, and using human responses to the prompts as positive (e.g., preferred) examples and finetuning by RLHF.

The exemplary systems, methods and computer-accessible medium according to exemplary embodiments of the present disclosure can further utilize embeddings of user queries from the chatbot to perform a similarity search over the EHR free text as a means of cohort discovery.

### Exemplary RLHF Process

Exemplary systems, methods and computer-accessible medium according to exemplary embodiments of the present disclosure can include and/or provide an exemplary procedure which can outline the process of using reinforcement learning from human feedback to align medical AI models with physicians and patients, referencing relevant EHR metadata.

### Exemplary Inputs:

EHR Data: Patient records containing relevant metadata such as demographics, medical history, lab results, and treatment outcomes.

Exemplary Feedback Interface: A user interface according to exemplary systems, methods and computer-accessible medium according to exemplary embodiments of the present disclosure can facilitate the physicians and patients to interact with the AI model and provide feedback on its recommendations or decisions.

### Exemplary Initialization:

Initialize AI Model: Exemplary systems, methods and computer-accessible medium according to exemplary embodiments of the present disclosure can train an initial AI model using, e.g., supervised learning techniques on historical EHR data to establish baseline performance.

Define Reinforcement Learning Components: Exemplary systems, methods and computer-accessible medium according to exemplary embodiments of the present disclosure can facilitate the setting up of components for RL from human feedback, including RLFD and DPO methods.

### Exemplary Loop:

While feedback is available perform the following exemplary procedures
(i) Present Exemplary Recommendations: Use the AI model according to exemplary systems, methods and computer-accessible medium according to exemplary embodiments of the present disclosure, to generate recommendations or decisions based on patient data,
(ii) Collect Exemplary Feedback: Present recommendations to physicians and patients via the feedback interface, and
(iii) Update Exemplary Policy.

### Exemplary Termination:

Exemplary Evaluation and Validation: Exemplary systems, methods and computer-accessible medium according to exemplary embodiments of the present disclosure can conduct validation studies using real-world data and/or user feedback to assess the effectiveness of the updated model.

Adjust Exemplary Parameters: Exemplary systems, methods and computer-accessible medium according to exemplary embodiments of the present disclosure can fine-tune model parameters based on performance metrics and user satisfaction.

Repeat Exemplary Loop: Exemplary systems, methods and computer-accessible medium according to exemplary embodiments of the present disclosure can be used and/or configured to iterate over the feedback collection loop, continuously improving the model's alignment with physicians and patients.

By integrating these components into the exemplary procedure, exemplary systems, methods and computer-accessible medium according to exemplary embodiments of the present disclosure can effectively leverage reinforcement learning from human feedback to align medical AI models with the expertise and preferences of physicians and patients, leading to more personalized and effective healthcare delivery. Human feedback can be the primary bottleneck of this training process, and therefore the exemplary systems, methods and computer-accessible medium according to exemplary embodiments of the present disclosure can replace human feedback by using weak models built upon EHR metadata.

### Exemplary Relevant EHR Metadata

The following EHR metadata can be relevant in implementing exemplary systems, methods and computer-accessible medium according to exemplary embodiments of the present disclosure.
Exemplary Patient Demographics: Age, gender, ethnicity, etc.
Exemplary Medical History: Previous diagnoses, surgeries, medications, allergies, etc.
Exemplary Laboratory Results: Blood tests, imaging reports, pathology results, etc.
Exemplary Treatment Outcomes: Response to medications, effectiveness of interventions, etc.
Exemplary Physician Annotations: Diagnosis correctness, relevance of treatment options, additional clinical notes, etc.
Patient Feedback: Satisfaction ratings, comments on recommendations, concerns or preferences regarding treatment plans, etc.

### Exemplary Generation of Exemplary Weak Model for AI Preference Feedback From Artifacts That can Approximate Human Preferences

The exemplary systems, methods and computer-accessible medium according to exemplary embodiments of the present disclosure can utilize artifacts (e.g., metadata, logs, timestamps, etc.) from the EHR and other clinical information systems to learn a model for human preferences that can provide “AI feedback” as part of RLAIF. This exemplary model, according to exemplary systems, methods and computer-accessible medium according to exemplary embodiments of the present disclosure, can have several forms depending upon the underlying use case. For example, an exemplary model for assessing the “quality of medical notes” can be rule-based with a mixed set of logical and natural language processing (NLP) rules such as, e.g.: (i) Check that datetime is less than 24 hours of a moving average of all notes to confirm note is not being written substantially later than typical, (ii) Check using regular expressions that the basic note structure follows SOAP format, and (iii) Check using billing criteria and NLP that codes match content.

Exemplary systems, methods and computer-accessible medium according to exemplary embodiments of the present disclosure can also model human preferences in a highly granular fashion using data more creatively. For example, the exemplary systems, methods and computer-accessible medium according to exemplary embodiments of the present disclosure can treat orders that have been changed or updated multiple times, or notes that have multiple edits, as surrogate signals for uncertainty or complexity. Likewise, the exemplary systems, methods and computer-accessible medium according to exemplary embodiments of the present disclosure can derive a signal for quality based on authorship, with trainee notes representing a lower degree of expertise (e.g., less preferred) than notes by more senior individuals. A simple join of authorIDs against faculty profiles can yield such a signal.

The exemplary systems, methods and computer-accessible medium according to exemplary embodiments of the present disclosure can perform the implicit modeling of text utilizing ground truth notes themselves from the EHR. With ground truth notes or messages from the EHR, the exemplary systems, methods and computer-accessible medium according to exemplary embodiments of the present disclosure can, e.g., directly, finetune models to approximate the language and thinking of physicians. Further, beyond supervised finetuning (SFT), the exemplary systems, methods and computer-accessible medium according to exemplary embodiments of the present disclosure can utilize physician generated text and direct preference optimization (DPO) in order to directly perform RLHF from the notes. Exemplary systems, methods and computer-accessible medium according to exemplary embodiments of the present disclosure can directly sample good and bad notes from the EHR, again, e.g., using the trainee status as a surrogate or the necessity for multiple edits, and with good and bad samples in hand DPO can be used to immediately align a given reward model with human preferences.

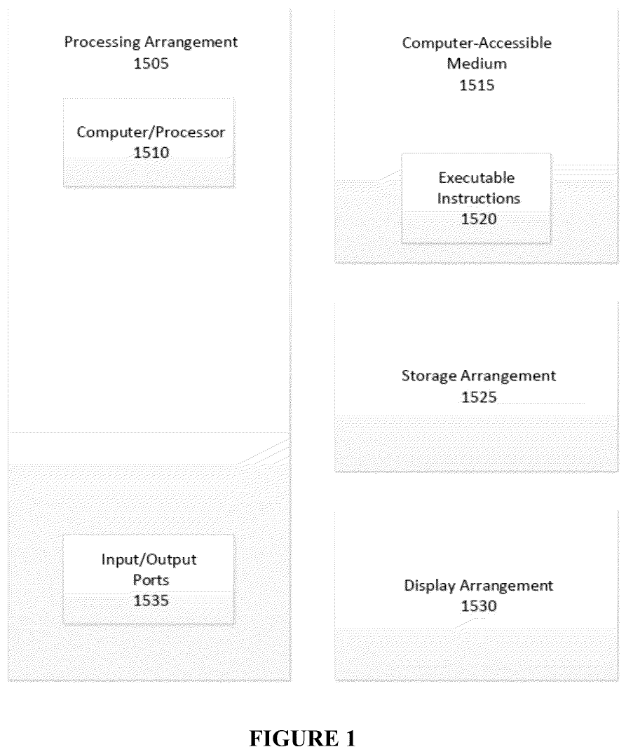

FIG. 1 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 1, for example, a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output ports 1535, which can include, for example a wired network, a wireless network, the internet, an intranet, a data collection probe, a sensor, etc. As shown in FIG. 1, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display arrangement 1530 and/or a storage arrangement 1525 can be used to display and/or store data in a user-accessible format and/or user-readable format.

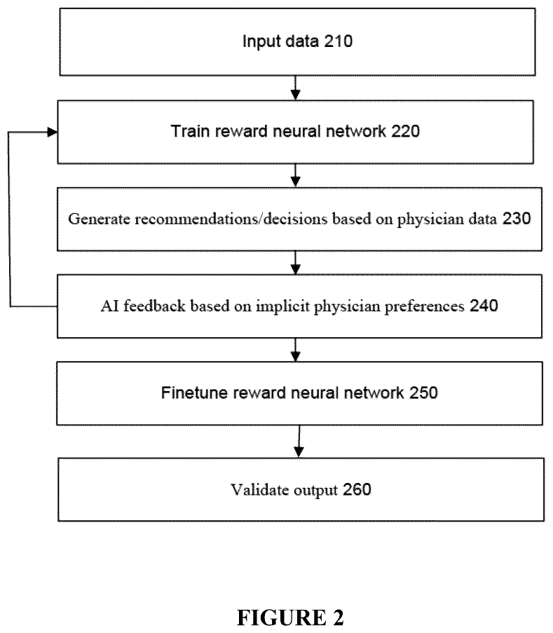

FIG. 2 illustrates a flow chart for a process of using reinforcement learning from AI on implicit physician preferences to align medical AI models with physicians and patients, referencing relevant EHR metadata, according to an exemplary embodiment of the present disclosure. For example, at step 210, data can be input into the reward neural network. The data can include, e.g., EHR, metadata, artifacts, and/or any other source of readily available physician created data. EHR data can include, for example, patient records containing relevant metadata such as demographics, medical history, lab results, and treatment outcomes.

At step 220, the reward neural network can be trained on the input data. The exemplary systems, methods and computer-accessible medium according to exemplary embodiments of the present disclosure can train an initial AI model using, e.g., supervised learning techniques on historical EHR data to establish baseline performance. At this step, reinforcement learning components can be defined. For example, the exemplary systems, methods and computer-accessible medium according to exemplary embodiments of the present disclosure can facilitate the setting up of components for reinforcement learning from AI feedback, including RLAIF and DPO methods. After initial training, the AI model can be further trained through a feedback interface.

At step 230, the trained neural network can generate recommendations or decisions based on EHR data, patient data, etc. At step 240, the generated recommendations and/or decisions can be presented to a surrogate AI model for evaluation and feedback. The surrogate AI model can use artifacts (metadata, logs, timestamps, etc.) from the EHR and other clinical information systems to infer physician preferences and compare that with the generated recommendations and/or decisions. Based on this comparison, the surrogate AI model can generate feedback for the reward neural network. At step 220, the reward neural network can be further trained with the feedback from the surrogate AI model.

At step 250, the reward neural network can be finetuned with ground truth notes or messages from the HER. In this exemplary manner, the reward neural network can be better trained to approximate the language and thinking of physicians. At step 260, output of the reward neural network can be validated. This validation can include, e.g., studies using real-world data and user feedback to assess the effectiveness of the updated model.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for training a language model for a medical use or performing a medically-related procedure, comprising:
modeling a reward neural network on one or more physician preferences; and
electronically training the language model by applying the reward neural network modeled on the one or more physician preferences as a feedback to the language model to guide the language model to learn the one or more physician preferences.

2. The method of claim 1, wherein the reward neural network relies on an artificial intelligence (AI) model as a surrogate reward function for the feedback.

3. The method of claim 2, wherein the physician preferences are obtained from the AI model providing the feedback for the one or more physician preferences.

4. The method of claim 3, wherein the modeling of the reward neural network comprises obtaining implicit physician preferences.

5. The method of claim 4, wherein the implicit physician preferences are derived from at least one of electronic health records, metadata, or artifacts.

6. The method of claim 5, wherein the implicit physician preferences are inferred from a physician written text provided in the at least one of the electronic health records, the metadata, or the artifacts.

7. The method of claim 1, wherein the reward neural network is finetuned on one or more ground truth notes in electronic health records to approximate the language and thinking of physicians.

8. A system for training a language model for a medical use or performing a medically-related procedure, comprising:
one or more computer processors configured to:
model a reward neural network on one or more physician preferences; and
electronically train the language model by applying the reward neural network modeled on the one or more physician preferences as a feedback to the language model to guide the language model to learn the one or more physician preferences.

9. The system of claim 8, wherein the reward neural network relies on an artificial intelligence (AI) model as a surrogate reward function for the feedback.

10. The system of claim 9, wherein the physician preferences are obtained from the AI model providing the feedback for the one or more physician preferences.

11. The system of claim 10, wherein the modeling of the reward neural network comprises obtaining implicit physician preferences.

12. The system of claim 11, wherein the implicit physician preferences are derived from at least one of electronic health records, metadata, or artifacts.

13. The system of claim 12, wherein the implicit physician preferences are inferred from a physician written text provided in the at least one of the electronic health records, the metadata, or the artifacts.

14. The system of claim 8, wherein the reward neural network is finetuned on one or more ground truth notes in electronic health records to approximate the language and thinking of physicians.

15. A non-transitory computer accessible medium which includes software thereon for training a language model for a medical use or performing a medically-related procedure, wherein, when at least one computer processor executes the software, the computer processor is configured to perform the procedures, comprising
modeling a reward neural network on one or more physician preferences; and
electronically training the language model by applying the reward neural network modeled on the one or more physician preferences as a feedback to the language model to guide the language model to learn the one or more physician preferences.

16. The computer accessible medium of claim 15, wherein the reward neural network relies on an artificial intelligence (AI) model as a surrogate reward function for the feedback.

17. The computer accessible medium of claim 16, wherein the physician preferences are obtained from the AI model providing the feedback for the one or more physician preferences.

18. The computer accessible medium of claim 17, wherein the modeling of the reward neural network comprises obtaining implicit physician preferences.

19. The computer accessible medium of claim 18, wherein the implicit physician preferences are derived from at least one of electronic health records, metadata, or artifacts.

20. The computer accessible medium of claim 15, wherein the implicit physician preferences are inferred from a physician written text provided in the at least one of the electronic health records, the metadata, or the artifacts.

21. The computer accessible medium of claim 8, wherein the reward neural network is finetuned on one or more ground truth notes in electronic health records to approximate the language and thinking of physicians.
